(19) United States
(12) Patent Application Publication
(10) Pub. No.: US 2002/0013264 A1
(43) Pub. Date: Jan. 31, 2002

(54) Neoglycan anticancer agents and uses thereof

(76) Inventors: Ralph D. Sanderson, Maumelle, AR (US); Carla Y. Pumphrey, Conway, AR (US)

Correspondence Address: Benjamin Aaron Adler, ADLER & ASSOCIATES, 8011 Candle Lane, Houston, TX, 77071, US

(21) Appl. No.: 09/921,032

(22) Filed: Aug. 2, 2001

Related U.S. Application Data
(63) Continuation-in-part of application No. 09/479,139, filed on Jan. 7, 2000.
(60) Provisional application No. 60/115,053, filed on Jan. 8, 1999.

(51) Int. Cl. A61K 38/00; A61K 31/728; A61K 31/727; A61K 31/726; A61K 31/721; A61K 31/737

(52) U.S. Cl. 514/2; 514/54; 514/56; 514/59

(57) ABSTRACT

The instant invention describes the production of neoglycans, compounds capable of inhibiting tumor cell growth. The heparan sulfate proteoglycan syndecan-1 is a tumor suppressor molecule that inhibits growth and induces apoptosis in several cancer cell lines. Attempts to create synthetic analogues of syndecan-1 by carbodiimide (EDAC) conjugation of a protein scaffold and GAG surprisingly revealed that the protein component is not required. Neoglycans consisting of EDAC-modified heparin and EDAC-modified chondroitin sulfate (CS), respectively named neoheparin and neo-chondroitin sulfate (neoCS), were found to inhibit multiple myeloma cell viability. Further analysis revealed the neoglycan compounds severely reduced cell viability of multiple myeloma, breast cancer and normal laboratory cell lines and peripheral blood mononuclear cells through the induction of apoptosis. Neoglycans provide a new class of GAG chain-based anticancer therapeutics.

### CROSS-REFERENCE TO RELATED APPLICATION

This non-provisional patent application is a continuation in part of U.S. Ser. No. 09/479,139, filed Jan. 7, 2000, which claims benefit of provisional patent application U.S. Ser. No. 60/115,053, filed Jan. 8, 1999, now abandoned.

### FEDERAL FUNDING LEGEND

This invention was created in part using funds obtained through grant NIH CA55819 from the National Institutes of Health and grants DAMD 17-99-1-9066 and DAMD 17-99-9064 from the United States Army. Consequently, the federal government h as certain rights in this invention.

### BACKGROUND OF THE INVENTION

1. Field of the Invention

The present invention relates generally to the fields of carbohydrate biochemistry and chemotherapy. More specifically, the present invention relates to novel, chemically modified glycosylaminoglycan molecules that induce apoptosis in cancer cells.

2. Description of the Related Art

The concept that long, unbranched carbohydrate polymer chains called glycosaminoglycans (GAG) can impact cancer progression has been investigated for decades. The GAG heparin may inhibit or stimulate tumor growth and metastasis in different animal models. In cancer patients, low molecular weight heparin treatment improves three month survival potentially by reducing angiogenesis (Folkman and Shing 1992; Siragusa, Cosmi et al. 1996; Bijsterveld, Hettiarachchi et al. 1999; Hejna, Raderer et al. 1999; Collen, Smorenburg et al. 2000; Smorenburg and Van Noorden 2001). Another glycosaminoglycan chain, hyaluronic acid (HA), inhibits melanoma tumor growth in an animal model but rescues and enhances proliferation of IL-6 dependant multiple myeloma cells cultured in the absence of exogenous IL-6 (Zeng, Toole et al. 1998; Vincent, Jourdan et al. 2001). Also, mutations that inhibit heparan sulfate polymerase enzymes cause hereditary multiple exostoses, which are characterized by the formation of benign, bone tumors (Lind, Tufaro et al. 1998; McCormick, Leduc et al. 1998).

GAG chains are attached to protein scaffolds, collectively forming proteoglycans, which are found on cell surfaces and in the ECM. Glycosaminoglycan chains mediate cell behaviors through the formation of GAG chain-ligand complexes. Variation in the specific characteristics of each GAG chain creates the diversity that is needed for binding a formidable number of ligands (Bernfield, Gotte et al. 1999; Park, Reizes et al. 2000; Turnbull, Powell et al. 2001). Interaction of ligands with GAG chains can be inhibitory or stimulatory and affects cell proliferation, differentiation, migration, and adhesion (Park, Reizes et al. 2000). Numerous factors, including the specific characteristics and functions of GAG chains and the type of cancer likely influence the effect of GAG chains on tumor cell proliferation and metastasis and therefore, cancer progression.

Cell surface heparan sulfate proteoglycans regulate cell behaviors by binding various growth factors, proteases, cell-matrix and cell-cell adhesion molecules. Through these interactions they mediate cell growth, survival, differentiation, adhesion and migration. Heparin and heparan sulfate bind and regulate ligands including but not exclusively, coagulation proteins, extracellular matrix (ECM) components, chemokines, cytokines, cell adhesion molecules, and numerous growth factors (Bernfield, Gotte et al. 1999). In most cells, the major source of cell surface heparan sulfate is syndecans.

The syndecans are a major family of cell surface heparan sulfate proteoglycans consisting of a core protein to GAGs, are covalently attached (Carey, Stahl et al. 1994). All four known members of the syndecan family (designated syndecan-1, -2, -3 and -4) are type I transmembrane proteins, with highly conserved transmembrane and cytoplasmic domains and an ectodomain that contains at least three sites for heparan sulfate attachment. Most of the syndecans' known functions occur via interaction of their -heparan sulfate chains with extracellular ligands and recent studies indicate that at least some syndecan cytoplasmic domains may b e directly involved in signaling (Reiland, Ott et al. 1996; Oh, Woods et al. 1997). Moreover, heparan sulfates are obligate partners for the high affinity signaling mediated by some growth factors (e.g. basic fibroblast growth factor, chemokines) (Rapraeger 1993; Tanaka, Kimata et al. 1998). Because they function in both growth control and cell adhesion, syndecans (and other heparan sulfate proteoglycans) play important roles in regulating the behavior of tumor cells (Inki and Jalkanen 1996).

An active form of syndecan-1 is shed from the cell surface by sheddases or secretases that release virtually the entire ectodomain including the attached heparan sulfate GAG chains (Arribas, Coodly et al.; Hooper, Karran et al. 1997). The syndecan-1 ectodomain suppresses the growth of CarB and S115, and MCF-7 mouse and human mammary tumor cells but not NIH3T3, NMuMG and HaCaT normal cell lines (Mali, Andtfolk et al. 1994). There is also an emerging correlation between cell surface syndecan-1 expression and cancer progression. Treatment of a multiple myeloma cell line and a plasma cell leukemia cell line with purified shed syndecan-1 ectodomain reduces cell growth and induces apoptosis (Dhodapkar, Abe et al. 1998). In addition, myeloma cells that are normally highly invasive in type I collagen gels lose their ability to invade following transfection with syndecan-1 (Liebersbach and Sanderson 1994). Normal mammary epithelial cells that are non-invasive on collagen gels become invasive when normal levels of cell surface syndecan-1 are lost (Kato, Saunders et al. 1995). Upon hormone-induced transformation of S115 mammary epithelia, these cells down-regulate their expression of syndecan-1, lose their epithelial morphology and become fusiform (Leppa, Harkonen et al. 1991). Following their transfection with the cDNA for syndecan-1, the transformed S115 cells regain their epithelial morphology and growth characteristics (Leppa, Mali et al. 1992). Similarly, when normal mammary epithelial cells are made deficient in syndecan-1 they lose their epithelial morphology and exhibit characteristics of neoplastically transformed cells including a fibroblastic appearance, the presence of elongated filopodial extensions and an absence of stress fibers and focal adhesions (Kato, Saunders et al. 1995).

In non-neoplastic lesions and metaplastic squamous cells of human uterine cervix, staining for syndecan was similar to that in normal uterine cervix (Inki, Joensuu et al. 1994). However, progression of cervical intraepithelial neoplasia from grade I to grade III was associated with a reduction in syndecan-l, and syndecan-1 was absent from poorly differentiated squamous cell carcinomas. In primary squamous cell carcinomas of the head and neck, tumors positive for syndecan-1 were associated with a more favorable patient prognosis. This prognosis included both a higher two year survival and a higher recurrence-free survival than those patients with tumors expressing little or no syndecan-1 (Inki, Joensuu et al. 1994). And in a more recent study of 100 patients with laryngeal squamous cell carcinoma, intermediate or strong staining for syndecan-1 was associated with higher overall survival than those tumors with little or no syndecan-1 expression (Pulkkinen, Penttinen et al. 1997).

Loss of syndecan-1 expression contributes to improved metastatic potential of non-small-cell lung and hepatocellular carcinomas (Inki, Joensuu et al. 1994; Matsumoto, Ono et al. 1997; Nackaerts, Verbeken et al. 1997; Pulkkinen, Penttinen et al. 1997; Kumar-Singh, Jacobs et al. 1998; Wiksten, Lundin et al. 2001). E-cadherin is known to suppress the invasion of numerous tumors including breast carcinoma (Mbalaviele, Dunstan et al. 1996). In mammary epithelia, loss of syndecan-1 is accompanied by a dramatic loss of E-cadherin expression (Kato, Saunders et al. 1995). In NM-c-ras-MAC1 mammary tumor cells, downregulation of E-cadherin with antisense RNA suppresses syndecan-1 expression and cells become invasive. Conversely, NM-f-ras-TD cells transfected with E-cadherin CDNA upregulate syndecan-l expression and become non-invasive (Leppa, Vleminckx et al. 1996).

Expression of syndecan-1 inhibits the growth of mammary epithelial tumor cells and loss of syndecan-1 on normal mammary epithelial cells promotes anchorage-independent growth, a hallmark of the neoplastic phenotype (Kato, Saunders et al. 1995). In myeloma cells, expression of syndecan-1 inhibits G1-S progression with downregulation of cyclin D1 (Dhodapkar, Abe et al. 1998). When injected into multiple myeloma cells in SCID mice, myeloma tumorigenesis is dramatically inhibited in cells transfected with the syndecan-1 cDNA as compared to vector-only transfected cells. This results in a reduction in disease related morbidity (Dhodapkar, Abe et al. 1998). Addition of 1-4 nM of exogenous, purified syndecan-1 ecctodomain inhibits the growth of both carcinoma (including S 115 and MCF-7 breast carcinoma) and myeloma cell lines (Mali, Andtfolk et al. 1994; Dhodapkar, Abe et al. 1998). Moreover, exogenous purified syndecan-1 induces a striking increase in apoptosis suggesting its potential usefulness for cancer therapy (Dhodapkar, Abe et al. 1998). These effects on growth require the intact proteoglycan because addition of either isolated heparan sulfate chains or core protein without heparan sulfate chains does not alter tumor cell growth.

These studies from three different laboratories using different cell lines and assay systems provide strong experimental evidence that syndecan-1 expressed on the cell surface or added exogenously can control tumor cell growth and motility. However, these growth inhibitory activities are not exclusive to syndecan-1 because glypican-1 and betaglycan, two other cell surface heparan sulfate proteoglycans, have similar effects on tumors (Lesoon-Wood, Kim et al. 1995). However, heparan sulfate proteoglycans are not present in large amounts on cells, it is difficult to harvest enough from natural sources to use for treatments. Therefore, it is important to make “synthetic” GAG-related molecules.

The prior art is deficient in the lack of synthetic proteoglycan and neoglycan compounds useful in inhibiting tumor cell growth. The present invention fulfills this long-standing need and desire in the art.

### SUMMARY OF THE INVENTION

To study the tumor suppressor functions of syndecan-1 and to develop syndecan-1 as a potential cancer therapeutic requires the isolation of large quantities of the heparan sulfate proteoglycan, a process that is severely hampered by the lack of a suitable system for the production of the protein bearing attached and appropriately modified heparan sulfate chains. To overcome this obstacle, synthetic proteoglycans that could potentially mimic syndecan-1 function were produced by linking GAG chains to human serum albumin (HSA) by carbodiimide (EDAC) conjugation. Treatment of multiple myeloma cell lines with the synthetic proteoglycans referred to as neoproteoglycans (nPGs) significantly reduced cell viability. However surprisingly, EDAC modified GAG chains also reduced multiple myeloma cell viability. This result indicates that a protein component is not necessarily required to produce a molecule with an anticancer effect. EDAC modified heparin and chondroitin sulfate (CS) were used for subsequent study and are termed neoglycans. In sharp contrast to native heparin and chondroitin sulfate, the neoglycans strikingly reduce cancer and normal cell viability, induce apoptosis, and the chondroitin sulfate neoglycan (neoCS) eliminates breast cancer tumor burden in a mouse model. Neoglycans are novel compounds and constitute a new class of anticancer therapeutic.

In one embodiment of the instant invention, anti-cancer agents designated as neoglycans are provided. Neoglycans are formed by chemically modifying GAGs with a carbodiimide such as 1-ethyl-3-(3-dimethylaminopropyl)carbodiimide (EDAC). The GAG chains may be heparin, heparan sulfate, chondroitin sulfate, hyaluronic acid, dextran or dextran sulfate. More specifically, the instant invention provides a neoglycan composed of EDAC modified heparin named neoheparin and a neoglycan composed of EDAC chondroitin sulfate chains named neo-chondroitin sulfate (neoCS).

Another embodiment of the instant invention is directed to a method producing neoglycans by treating a solution of a GAG with a carbodiimide solution for a period of time. The reaction is then subjected to centrifugation to remove any precipitate. The resulting supernatant is buffer exchanged into water. Preferably the method uses 1-ethyl-3-(3-dimethylaminopropyl)carbodiimide (EDAC) as the carbodiimide and heparin, heparan sulfate, chondroitin sulfate, hyaluronic acid, dextran and/or dextran sulfate as the glycosylaminoglycan.

Another embodiment of the instant invention is a pharmaceutical composition comprising a neoglycan and a pharmaceutically acceptable carrier.

Yet another embodiment of the instant invention is a method of inhibiting tumor growth by exposing a tumor to a pharmaceutical composition containing a neoglycan. Specific tumors treatable with the composition of the instant invention include breast cancer, multiple myeloma and others.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1H show that neoglycans reduce cell liability in a dose-dependent manner.

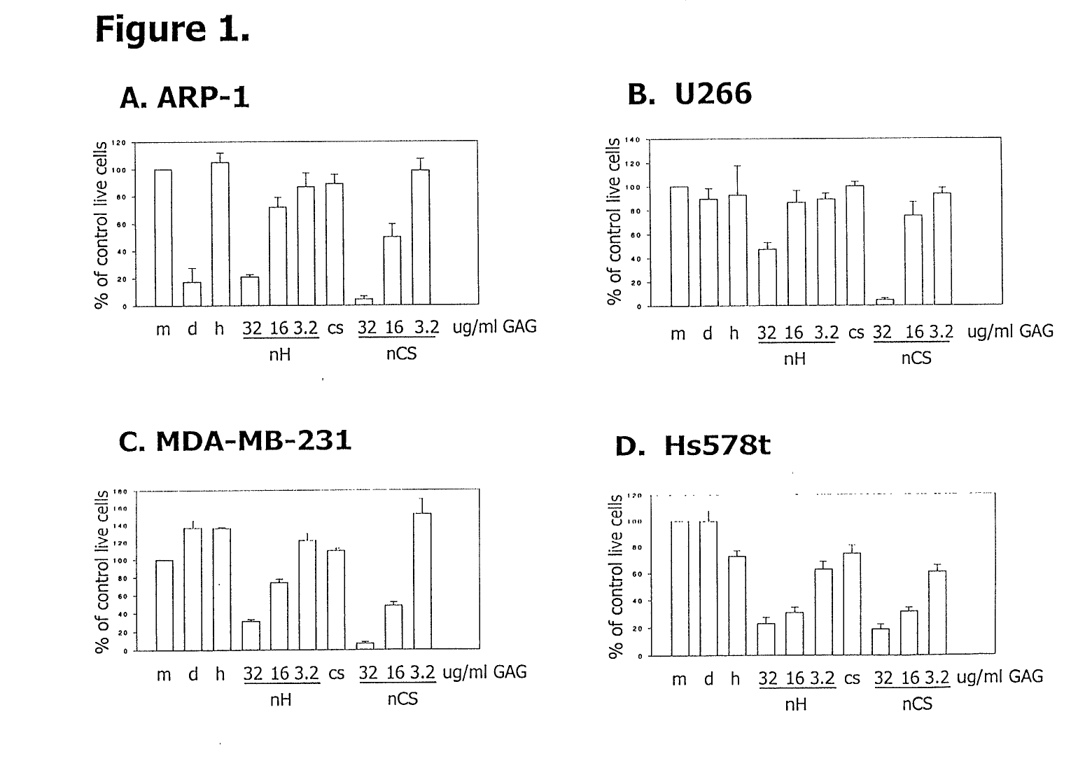

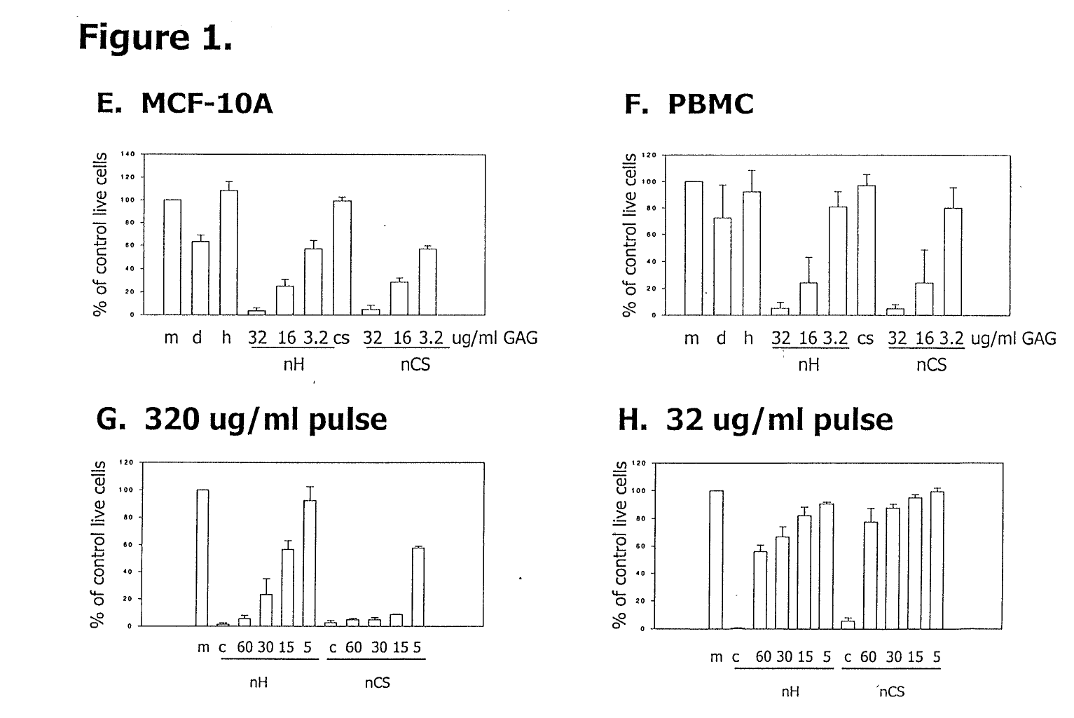

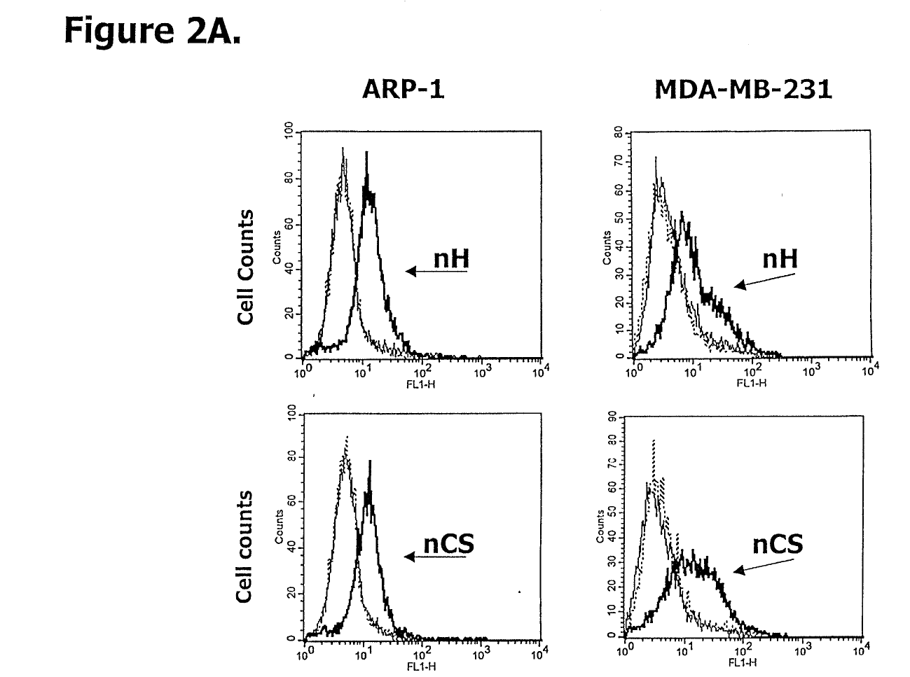

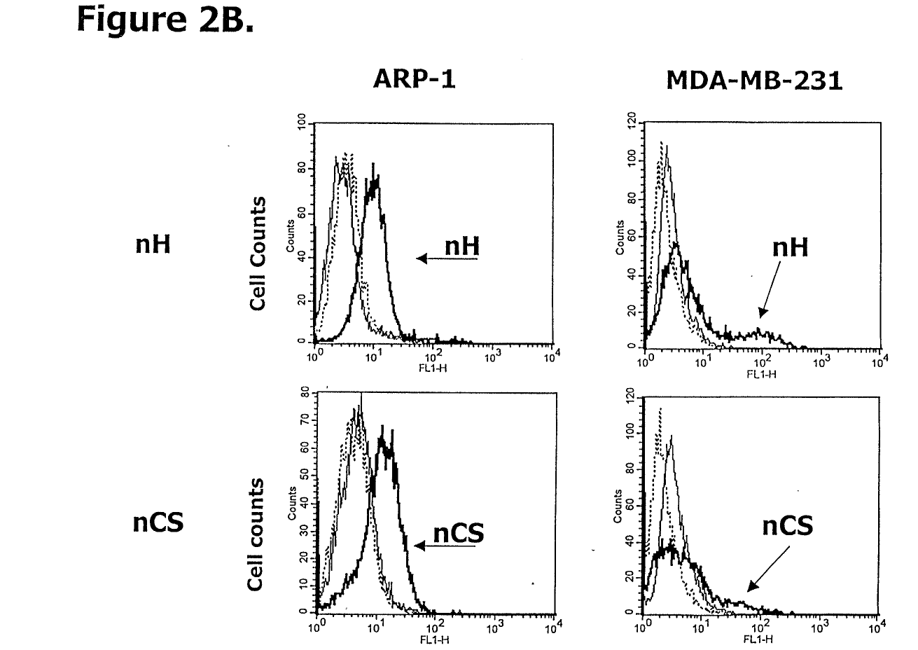

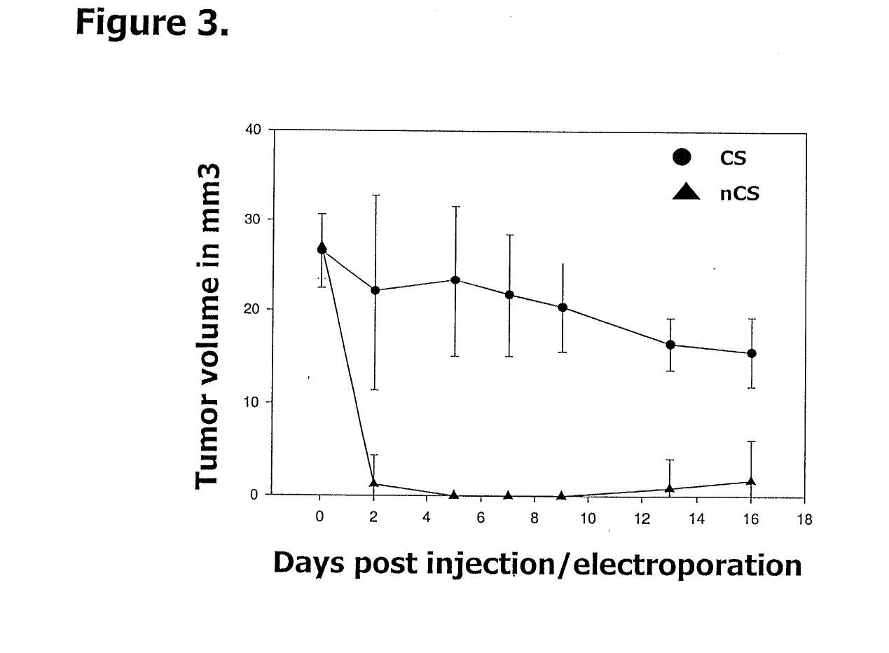

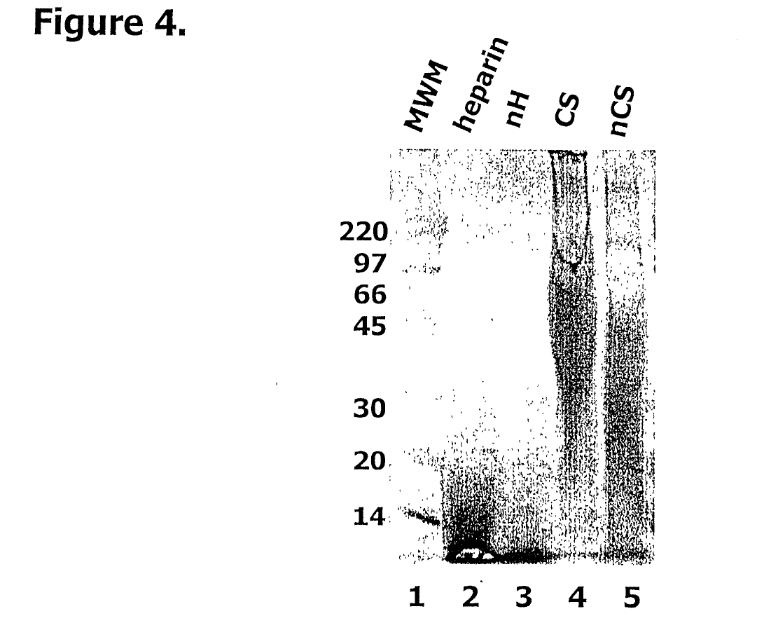

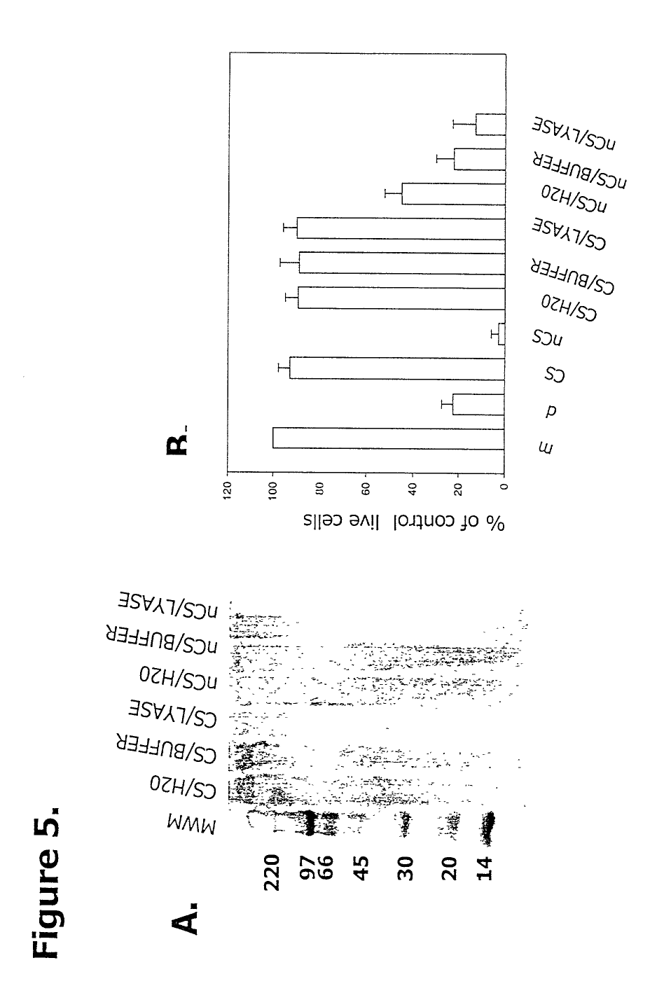

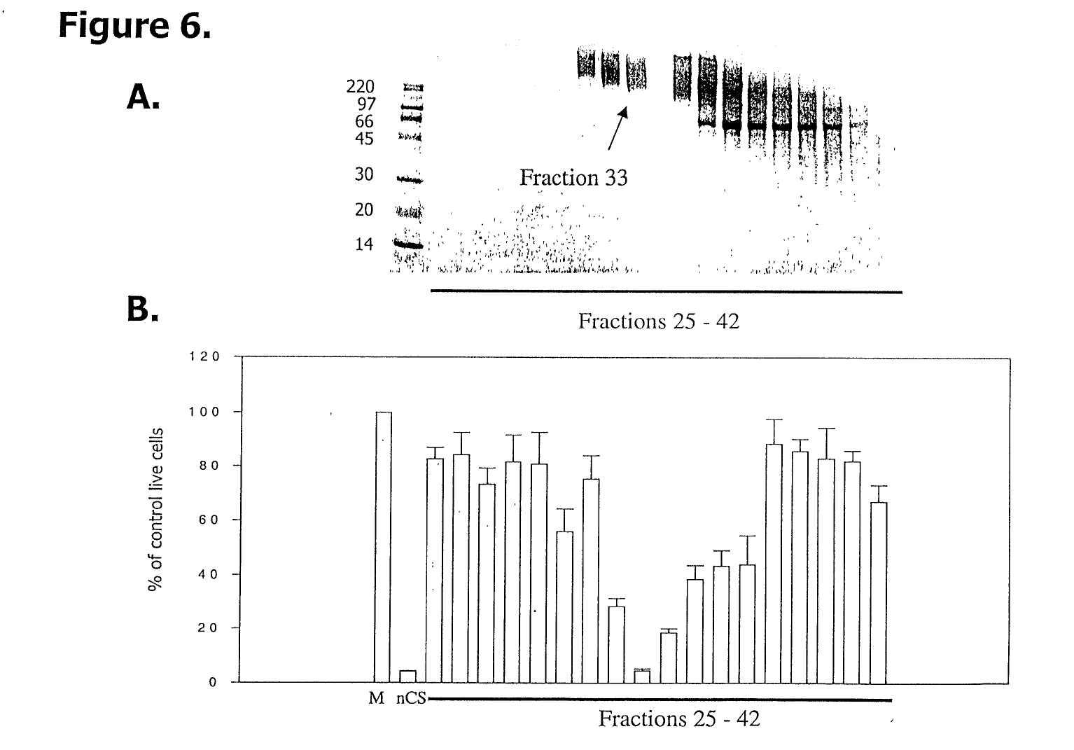

In FIGS. 1A-1F, triplicate wells of each cell line were cultured for 72 hours in the continuous presence of 32, 16, or 3.2 μg/ml concentrations of neoglycans in media containing only media (m), 10 μM dexamethasone (d), 500 μg/ml heparin (h) or 500 μg/ml chondroitin sulfate (cs). Cell viability was determined by the MTT assay and the results show the mean±standard deviations of duplicate experiments.

In FIGS. 1G and 1H, ARP-1 cells were treated with media only or neoglycan's for 72 hours as controls (c) or with 320 μg/ml nG's (FIG. 1G) or 32 μg/ml neoglycans (FIG. 1H) for 60, 30, 15 or 5 minutes. The cells were then washed and plated in triplicate. The MTT assay to determine cell viability was performed after 72 hours and the results are shown as means±standard deviation of duplicate experiments. In each experiment, the media only treatment indicates 100% cell viability.

FIGS. 2A and 2B show that neoglycans induce apoptosis. In FIG. 2A, ARP-1 multiple myeloma and MDA-MB-231 breast cancer cells were exposed to 32 μg/ml heparin or chondroitin sulfate (thin lines), neoheparin or chondroitin sulfate neoglycan (thick lines) or media only (dashed lines). Apoptotic cells were detected by flow cytometry based on Annexin-FITC staining and PI exclusion. FIG. 2B shows that DNA laddering, indicative of the late stages of apoptosis, was detected by the TUNEL assay. ARP-1 and MDA-MB-231 cell lines were treated with media (dashed line), 32 μg/ml heparin or chondroitin sulfate (thin line) or 32 μg/ml neoglycans (thick lines).

FIG. 3 shows that NeoCS eliminates established MDA-MB-231 tumors. Established MDA-MB-231 tumors were injected with a single dose of either 1.6 mg chondroitin sulfate or 1.6 mg chondroitin sulfate neoglycan followed by electroporation of the tumors. At the indicated time points, tumor size was measured and tumor volume with standard deviation was calculated.

FIG. 4 shows the visualization of neoglycans and GAG chains. Neoglycans (neoheparin and neoCS) and the GAG chains heparin (h) and chondroitin sulfate were separated by SDS-PAGE under reducing conditions and visualized following alcian blue staining. Molecular weight markers are shown in lane 1. Numeric values indicate molecular weights in kDa.

FIGS. 5A and 5B shows that an active fraction of chondroitin sulfate neoglycan is resistant to enzyme digestion. In FIG. 5A, chondroitin sulfate and chondroitin sulfate neoglycan were suspended in H20 (H), chondroitin ABC lyase buffer (B) or chondroitin ABC lyase (L) for several hours and a portion w as separated by SDS-PAGE and visualized following alcian blue staining. Molecular weight markers are shown in lane 1, and the size of each is indicated in kDa.

FIG. 5B shows triplicate wells of ARP-1 cells that were exposed to media only (m), 10 μM dexamethasone (d), 32 μg/ml chondroitin sulfate or neoCS, and pretreatment with equivalent amounts of chondroitin sulfate or chondroitin sulfate neoglycan in H₂O, lyase buffer, or chondroitin ABC lyase. Following a 72 hour incubation, cell viability was determined by the MTT assay. The results are expressed as the % viable cells relative to media only treated cells and the standard deviation for each condition is shown.

FIGS. 6A and 6B show the identification of active chondroitin sulfate neoglycan fractions. Chondroitin ABC lyase digested chondroitin sulfate neoglycan was fractionated by size exclusion chromatography and the guanidine removed by buffer exchange using spin column filtration. Equivalent amounts of each fraction were visualized on SDS-PAGE gels by alcian blue staining (FIG. 6A) Molecular weight markers are included in lane 1 and the size of each standard is indicated in kDa. Fractions 25 through fraction 42 are shown.

In FIG. 6B, equivalent amounts of each fraction were added to triplicate wells of ARP-1 cells in culture which were incubated for 72 hours, and % cell viability was determined by the MTT assay. Media only treatment (m) indicates 100% cell viability and chondroitin sulfate neoglycan treatment demonstrates the usual reduction in cell viability. Cell viability following treatment with the chondroitin sulfate neoglycan fractions 25-42 is shown and standard deviation is reported.

### DETAILED DESCRIPTION OF THE INVENTION

Heparan sulfate proteoglycans on the cell surface act a s tumor suppressors. When the syndecan-1 proteoglycan is lost from the surface of normal mammary epithelia, the cells lose their epithelial morphology, invade collagen gels and exhibit characteristics of neoplastic growth. When transfected with the cDNA for syndecan-1, transformed mammary epithelial cells regain their epithelial morphology and lose their neoplastic growth characteristics. Remarkably, addition of purified intact syndecan-1 ectodomain to tumor cells also inhibits their growth in culture and can induce extensive apoptosis. These effects require the intact proteoglycan because addition of either isolated heparan sulfate chains or core protein without heparan sulfate chains does not alter tumor cell growth. However, anti-tumor effects are not specific for syndecan-1, because other cell surface heparan sulfate proteoglycans (e.g. glypican-1 and betaglycan) also inhibit tumor cell growth.

The instant invention provides anti-cancer agents designated as neoglycans. Neoglycans formed by chemically modifying glycosaminoglycans with a carbodiimide. Preferably, the carbodiimide is 1-ethyl-3-(3-dimethylaminopropyl)carbodiimide (EDAC) and the glycosaminoglycan chains may be heparin, heparan sulfate, chondroitin sulfate, hyaluronic acid, dextran or dextran sulfate. More specifically, the instant invention provides a neoglycan composed of EDAC modified heparin named neoheparin and a neoglycan composed of EDAC chondroitin sulfate chains named neo-chondroitin sulfate (neoCS).

The instant invention is also directed to a method producing the neoglycans by treating a solution of a glycosaminoglycan with a carbodiimide solution. The reaction is then subjected to centrifugation to remove any precipitate. The resulting supernatant is then buffer exchanged into water. Preferably the method uses is 1 -ethyl-3-(3-dimethylaminopropyl)carbodiimide (EDAC) as the carbodiimide and heparin, heparan sulfate, chondroitin sulfate, hyaluronic acid, dextran and/or dextran sulfate as the gfiycosylaminoglycan.

The instant invention is also directed to a pharmaceutical composition comprising a neoglycan and a pharmaceutically acceptable carrier.

The instant invention also provides a method of inhibiting tumor growth by exposing a tumor to a pharmaceutical composition containing neoglycans. Specific tumors that can b e treated with this method include breast cancer, multiple myeloma and others.

The following examples are given for the purpose of illustrating various embodiments of the invention and are not meant to limit the present invention in any fashion. GAG

### EXAMPLE 1

Neoglycan Production

Neoglycans were prepared from porcine intestinal heparin and chondroitin sulfate C from shark cartilage (Sigma, St Louis, Mo.). A 5 mg/ml solution of GAG chains in deionized H20 w as brought to a 0.1 M concentration of 1-ethyl-3-(3-dimethylaminopropyl)carbodiimide (EDAC). The mixture was rocked overnight at 4° C. The reaction mixture was centrifuged at 20,000 g for 10 minutes to pellet any precipitant. The supernatant was collected and exhaustively buffer exchanged into H₂O and concentrated using 5,000 kDa molecular weight cut off spin columns (Millipore, Bedford, Mass.). Neoglycan preparations were stored at 4° C. Prior to in vivo studies, chondroitin sulfate neoglycan was brought to 0.15 M NaCl.

### EXAMPLE 2

Neoglycan Concentration Determination by Carbazole Assay

Carbazole assays were performed according to the protocols of Blumenkrantz and Asboe-Hansen (1973) and Filisetti-Cozzi and Carpita (1991) (Blumenkrantz and Asboe-Hansen 1973; Filisetti-Cozzi and Carpita 1991). Known concentrations of heparin and CS ranging from 0-60 μg were aliquoted into glass test tubes. One μl of each neoglycan, approximately 50 μg, was aliquoted into glass test tubes in duplicate. The volume of each tube was brought to 200 μl with H₂O. A single ml of very cold 125 mM sodium tetraborate (Sigma, St Louis, Mo.) in sulfuric acid was added to each tube followed by the addition of 35 μl of a carbazole reagent composed of 0.125% carbazole (Sigma, St. Louis, Mo.) w/v in ETOH. The tubes were mixed carefully and heated to 100° C. for 20 minutes. Cooled aliquots of each were transferred to a 96-well plate and the absorbance at OD₅₄₀ was measured on a microplate reader. Known GAG chain concentrations served as a standard curve for the extrapolation of the unknown neoglycan concentrations.

### EXAMPLE 3

Cells and Cell Culture

ARK, ARP-1, and CAG cells are B lymphoid cell lines established at the Arkansas Cancer Research Center from bone marrow aspirates of multiple myeloma patients (Ridley, Xiao et al. 1993). U266 is a multiple myeloma cell line that was obtained from the American Type Culture Collection (ATCC, Manassas, Va.). ARH-77 cells (ATCC, Manassas, Va.) were established from the peripheral blood of a patient with plasma cell leukemia (Burk, Drewinko et al. 1978). Peripheral blood mononuclear cells (PBMCs) were collected from healthy donors by Ficol-Hypaque separation of whole blood and were mitogen activated for 3 days with 2.5 μg/ml PHA (Sigma, St Louis, Mo.) prior to neoglycan treatment. The cell lines and peripheral blood mononuclear cells were grown in RPMI 1640 supplemented with 10% fetal bovine serum, 2 mM L-glutamine, 1 00 U/ml penicillin and 100 μg/ml streptomycin sulfate. HBL-100, MCF-7, MDA-MB-231, MDA-MB-435, and MDA-MB-436 breast cancer cell lines were obtained from ATCC and were maintained in MEM containing 10% fetal bovine serum, 2 mM L-glutamine, 100 U/ml penicillin, 100 μg/ml streptomycin sulfate, and esterdiol. The Hs578t breast cancer cell line (ATCC, Manassas, Va.) was grown in DMEM supplemented with 10% FBS, 100 U/ml penicillin, 100 μg/ml streptomycin sulfate, 1 mg/100 ml insulin, 1 mM sodium pyruvate, and 1.5 g/L NaHCO₃. Normal cell lines including CHO-K1 Chinese hamster ovary cells, NIH 3T3 Swiss murine embryo fibroblast cells, NMuNG murine mammary cells, MDCK canine kidney cells, and Hs578Bst, MCF-10A, and MCF-10F human mammary cell lines were all purchased from the ATCC. CHO-K1 cells were cultured in F12K media containing 10% fetal bovine serum, 2 mM L-glutamine and 1.5 g/L NaHCO₃. The NIH 3T3 cell line was grown in DMEM supplemented with 10% fetal bovine serum, 4 mM L-glutamine, 4.5 g/L glucose, and 1.5 g/L NaHCO₃. NMuNG cells were grown in DMEM containing 10% fetal bovine serum, 2 mM L-glutamine, 4.5 g/L glucose and 10 μg/ml insulin. The MDCK cell line was cultured in EMEM containing 10% fetal bovine serum, 1.0 mM sodium pyruvate, 0.1 mM nonessential amino acids, and 1.5 g/L NaHCO₃. The Hs578Bst cells were grown in Hybri-Care media (ATCC, Manassas, Va.) containing 10% fetal bovine serum, 100 U/ml penicillin, 100 μg/ml streptomycin sulfate, and 1.5 g/L NaHCO₃. The MCF-10A and MCF-10F cells were cultivated in MEM supplemented with 10% fetal bovine serum and 100 ng/ml cholera toxin.

### EXAMPLE 4

Determination of Cell Viability by the MTT Assay

Cells were plated in the appropriate media on 96-well plates in a 100 μl total volume. Non-adherent cells were plated at a density of 2×10⁴ cells/well. Adherent cell density varied based on the growth characteristics of each cell line and these cells were plated one day prior to neoglycan addition to allow the cells to attach. Triplicate wells were treated with media, 10 μM dexamethasone, 50 μg heparin or CS, and 3.2 μg, 1.6 μg, or 0.32 μg of neoheparin or neoCS. The plates were incubated at 37° C. in 5% CO₂ for 72 hours. Cell viability was determined based mitochondrial conversion of 3[4,5-Dimethylthiazol-2-yl]-2,5-diphenyltetrazolium bromide (MTT, Sigma, St Louis, Mo.) to formazan. The amount of MTT converted to formazan is indicative of the number of viable cells (Pieters, Huismans et al. 1988). Each well was supplemented with 50 μof a 2 mg/ml solution of MTT in complete media. The plates were returned to 37° C. and 5% CO₂ for 4-5 hours. The media was carefully removed from each well and 150 μl of 37° C. DMSO was added. The plates were gently agitated until the color reaction was uniform and the OD₅₄₀ was determined using a microplate reader. SigmaPlot, 2000 software was used for data analysis. Media only treated cells served as the indicator of 100% cell viability.

To determine if continuous exposure to neoglycans is required for growth inhibition, ARP-1 cells were exposed to 320 μg/ml or 32 μg/ml neoglycans for 5, 15, 30, and 60 minutes. The cells were washed twice in complete media, plated in triplicate and incubated at 37° C., 5% CO₂ for 72 hours. A control was included in which the cells were treated with the neoglycans for the usual 72 hours. Cell viability was determined by MTT assay as described above.

### EXAMPLE 5

Apoptosis Determination

ARP-1 cells and MDA-MB-231 cells were treated with media only, 32 μg/ml GAG chains and 32 μg/ml neoglycans. The ARP-1 cells were harvested following a 48 hour incubation period and the MDA-MB-231 cells were harvested after a 96 hour incubation period. The induction of apoptosis was determined by staining with fluorescein isothiocyanated-labeled Annexin V (Annexin V-FITC, CALTAG Laboratories, Burlingame, Calif.) following the manufacturer's instructions. Just prior to analysis, 0.5 mg/ml propidium iodide (PI, Sigma, St Louis, Mo.) in PBS, pH. 7.4 was added. Flow cytometry was performed with a Beckman Dickenson (Mountain View, Calif.) FACScan using Cellquest 1.2 software. Annexin V-FITC positive, PI negative cells were considered apoptotic.

To evaluate a late event of apoptosis, DNA ladder formation, TUNEL assays were performed following treatment of ARP-1 and MDA-MB-231 cells as described above. ARP-1 and MDA-MB-231 cells were harvested following a 48 hour or a 120 hour incubation period, respectively. Apoptotic cells were identified b y TdT-mediated dUTP nick end labeling (TUNEL assay) using the Roche Molecular Biochemicals Fluorescein In Situ Cell Death Detection Kit (Boehringer Mannheim, Indianapolis, Ind.) according to the manufacturer's instructions. The cells were analyzed by FACscan using Cellquest 1.2 software.

### EXAMPLE 6

Treatment of Established MDA-MB-231 Tumors with CS and neoCS.

BALB/c nu/nu mice were anesthetized by intraperitoneal administration of a mixture of ketamine (42.8 mg/ml), xylazine (8.6 mg/ml), and acepromazine (1.4 mg/ml) at a dose of 1.8-2.0 ml/kg. Human breast cancer tumors were established following subcutaneous injection of 2×10⁶ MDA-MB-231 cells in PBS. Two tumors were established on each mouse. Tumors were injected with a single 1.6 mg dose of chondroitin sulfate or chondroitin sulfate neoglycan in a 50 μl total volume. Immediately following injection, a caliper electrode was positioned on t wo sides of the tumor with contact on the skin. An electrical pulse was applied to the tumor through the electrodes with a BTS EC830 power supply (Genetronics, Inc., San Diego, Calif.). The tumors were measured with calipers and tumor volumes calculated according to the equation: V=(L×W2)×0.5. The results reflect two separate experiments in which one tumor was treated with chondroitin sulfate and the second tumor on the same animal was treated with neoCS. Both experiments included 7 tumors per treatment group.

### EXAMPLE 7

SDS-PAGE Analysis of Neoglycans

GAG chains and neoglycans were separated by SDS-PAGE through 14% gels. The gels were first stained with 0.25% coomassie blue for several hours and destained in methanol:H₂O (1:1 v/v) and 10% glacial acetic acid overnight. The gels were subsequently stained with 0.1% alcian blue, 3% glacial acetic acid for 15 minutes and destained in H₂O. Rainbow molecular weight markers (Amersham Pharmacia biotech, Piscataway, N.J.) served as size standards on each gel.

### EXAMPLE 8

Polysaccharide Lyase Digestion and Superdex 200 Column Chromatography of Neoglycans

Chondroitin sulfate and chondroitin sulfate neoglycan (500 μg each) were speed vacuumed to dryness and resuspended in H₂O, 20 mM Tris-HCl, pH. 8.0 (lyase buffer), or 150 μU chondroitin ABC lyase (Segagiku, Tokyo, Japan). Digestions were performed at 37° C. for 4 hours. Superdex 200 prep grade gel filtration media (Amersham Pharmacia Biotech, Piscataway, N.J.) which fractionates proteins by size in a range from 10-600 kDa was equilibrated in 6 M guanidine, 50 mM sodium acetate, pH. 5.8 column running buffer. A 95 cm×2 cm Superdex 200 column was poured and packed at a flow rate of 6 ml/hour. The column was washed with one bed volume of funning buffer. A 100 mg preparation of chondroitin sulfate neoglycan was digested with 0.3 U of chondroitin ABC lyase in 20 mM Tris, pH 8.0 for 4 hours. The digested chondroitin sulfate neoglycan was brought 1:1 with column running buffer, boiled for 10 minutes, cooled to room temperature, loaded on the Superdex 200 column, and fractions were collected. Numerous fractions were buffer exchanged into H₂O by 5,000 molecular weight cut off spin column filtration (Millipore, Bedford, Mass.) and stored at 4° C.

### EXAMPLE 9

Neoglycans Reduce Cell Viability in vitro

Synthetic proteoglycans termed neoproteoglycans (nPG), were produced by EDAC conjugation of the GAG chains heparin or chondroitin sulfate and a protein scaffold. Treatment of a multiple myeloma cell line with either nPG preparation or controls produced by EDAC modification of GAG chains alone reduced cell viability (data not shown). Native heparin and chondroitin sulfate, and protein coupled to protein had no growth reducing affect on multiple myeloma cells. These results indicate that protein is not necessary for the production of a molecule that reduces multiple myeloma cell viability. Therefore, neoglycans (neoglycans) consisting of EDAC modified heparin (neoheparin) or chondroitin sulfate (neoCS, neoCS) were produced and evaluated for potential anticancer activities.

The concentrations of the neoheparin and chondroitin sulfate neoglycan preparations were determined by a carbazole assay using native heparin and chondroitin sulfate as standards. The effect of GAG chains and neoglycans on cell viability was tested on several cancer cell lines including the multiple myeloma cell lines ARK, ARP-1, CAG and U266; a plasma cell leukemia cell line ARH-77; and, breast cancer cell lines Hs578t, MCF-7, MDA-MB-231, MDA-MB-435 and MDA-MB-436. Dexamethasone is a known inducer of apoptosis in some multiple myeloma cell lines including the ARP-1 cell line (Alexanian, Barlogie et al. 1986) and is included for comparison and as a positive control for the reduction of cell viability. The results of the MTT assays demonstrate a remarkable dose-dependant reduction in cancer cell viability in response to treatment with either neoheparin or chondroitin sulfate neoglycan (FIGS. 1A-1D). Neoheparin and chondroitin sulfate neoglycan (32 μg/ml) produce a 79% and 96% reduction in ARP-1 multiple myeloma cell viability, respectively.

In comparison, heparin treatment slightly enhances cell proliferation and chondroitin sulfate has no affect. The 50% inhibitory concentration (IC₅₀) values for neoheparin and chondroitin sulfate neoglycan on ARP-1 cells are 21.94 μg/ml (95% confidence interval [CI] of 18.18 to 26.48 μg/ml) and 14.79 μg/ml (95% CI of 12.13 to 18.06 μg/ml), respectively (Table 1). U266 multiple myeloma cells exhibit similar sensitivity to chondroitin sulfate neoglycan (IC₅₀=19.05 μg/ml, 95% CI of 16.83 to 21.55 μg/ml) but are less susceptible to treatment with neoheparin (IC₅₀=32.00 μg/ml, 95% CI of 26.44 to 38.73 μg/ml; FIG. 1B and Table 1) Comparison of the titration of the neoglycans on the MDA-MB-231 breast cancer cell line indicates that these cells are more sensitive to chondroitin sulfate neoglycan than neoheparin (FIG. 1C and Table 1). The IC₅₀ value for chondroitin sulfate neoglycan is 5.00 μg/ml with a 95% CI of 3.28 to 7.61 μg/ml whereas the IC₅₀ value for neoheparin is 17.70 μg/ml, 95% CI of 14.59 to 21.28 μg/ml. The amount of neoheparin needed to reduce Hs578t breast cancer cell viability by 50% is 2.34 μg/ml, 95% CI of 1.67 to 3.28 μg/ml (FIG. 1D, Table 1). NeoCS treatment shows similar results with an IC₅₀ concentration of 3.65 μg/ml, 95% CI of 3.20 to 4.16 μg/ml.

Identical experiments were performed to evaluate the affect of the neoglycans on normal cell lines including: CHO-K1, MDCK, NIH 3T3, NMuNG, Hs578Bst, MCF-10A, MCF-10F, HBL-100 human breast cell line, which is not tumorigenic at low passage numbers but is tumorigenic at high passage numbers, and primary peripheral blood mononuclear cells. The results demonstrate that the normal cell lines tested are sensitive to treatment with either neoglycan (FIGS. 1E-1F, Table 1).

TABLE 1
Fifty percent inhibitory concentration of neoglycans
for the reduction of cancer and normal cell viability in vitro.

| | | | | | nH | nH CI^{b} | nCS | nCS CI^{b} |
|---|---|---|---|---|---|---|---|---|
| | | | | | IC50^{a} | range | IC50^{a} | range |
| Organism | Tumorgenicity | Tissue | Morphology | Designation | (ug/ml) | (ug/ml) | (ug/ml) | (ug/ml) |
| human | + | multiple | lymphoblast, | ARK | 27.42 | 21.96 | 24.92 | 18.03 |
| | | myeloma | plasma cell | | | 34.24 | | 34.45 |
| human | + | multiple | lymphoblast, | ARP-1 | 21.94 | 18.18 | 14.79 | 12.13 |
| | | myeloma | plasma cell | | | 26.48 | | 18.06 |
| human | + | multiple | lymphoblast, | CAG | 16.02 | 14.09 | 13.65 | 11.88 |
| | | myeloma | plasma cell | | | 18.21 | | 15.68 |
| human | + | multiple | lymphoblast, | U266 | 32.00 | 26.44 | 19.05 | 16.83 |
| | | myeloma | plasma cell | | | 38.73 | | 21.55 |
| human | + | plasma cell | lymphoblast, | ARH-77 | 19.74 | 12.18 | 21.87 | 9.06 |
| | | leukemia | plasma cell | | | 31.99 | | 52.80 |
| human | ± | breast | epithelial | HBL-100 | 8.28 | 5.89 | 6.81 | 2.92 |
| | | adenocarcinoma | | | | 11.65 | | 15.90 |
| human | + | breast | epithelial | Hs578t | 2.34 | 1.67 | 3.65 | 3.20 |
| | | adenocarcinoma | | | | 3.28 | | 4.16 |
| human | + | breast | epithelial | MCF-7 | 11.49 | 3.52 | 6.55 | 4.26 |
| | | adenocarcinoma | | | | 37.49 | | 10.06 |
| human | + | breast | epithelial | MDA-MB-231 | 17.70 | 14.59 | 5.00 | 3.28 |
| | | adenocarcinoma | | | | 21.28 | | 7.61 |
| human | + | breast | epithelial | MDA-MB-435 | 17.10 | 12.25 | 6.82 | 4.51 |
| | | adenocarcinoma | | | | 23.86 | | 10.29 |
| human | + | breast | epithelial | MDA-MB-436 | 4.21 | 2.66 | 3.86 | 2.80 |
| | | adenocarcinoma | | | | 6.68 | | 5.33 |
| human | − | breast | epithelial | MCF-10A | 4.16 | 2.76 | 4.29 | 2.67 |
| | | | | | | 6.29 | | 6.89 |
| human | − | breast | epithelial | MCF-10F | | | | |
| chinese | − | ovary | epithelial | CHO-K1 | 3.38 | 2.23 | 3.33 | 3.13 |
| hamster | | | | | | 5.13 | | 3.53 |
| human | − | breast | epithelial | Hs578BST | | | | |
| canine | − | kidney | epithelial | MDCK | 15.98 | 4.97 | 10.64 | 7.63 |
| | | | | | | 51.36 | | 14.83 |
| murine | − | embryo | epithelial | NIH3T3 | 7.73 | 3.42 | 15.74 | 12.22 |
| | | | | | | 17.50 | | 20.29 |
| murine | − | breast | epithelial | NMuNG | 6.29 | 4.26 | 5.87 | 4.60 |
| | | | | | | 9.29 | 7.50 | |
| human | − | blood | lymphocyte, | PBMC | 3.94 | 3.22 | 4.21 | 3.64 |
| | | | monocyte | | | 4.82 | 4.86 | |

^{a}Estimated IC50 values were determined following cell viability assays by use of a compound nonlinear degradation model based on the 3 parameter logistic. Calculations are based on triplicat wells from duplicate experiments.
^{b}95% confidence interval.

Treatment of the MCF-10A cell line with 4.16 μg/ml neoheparin (95% CI of 2.76 to 6.29 μg/ml) or 4.29 μg/ml chondroitin sulfate neoglycan (95% CI of 2.67 to 6.89) reduces cell viability by 50% (FIG. 1E and Table 1). Mitogen activated peripheral blood mononuclear cells showed similar sensitivity to both neoglycans (FIG. 1F and Table 1) with C50 values of 3.94 μg/ml for neoheparin (95% CI of 3.22 to 4.82 μg/ml) and 4.21 for chondroitin sulfate neoglycan (95% CI of 3.64 to 4.86 μg/ml). Therefore, the viability of multiple myeloma, breast cancer and normal cell lines, and activated peripheral blood mononuclear cells is reduced by neoglycan administration.

To determine if the continuous presence of the neoglycan is is required for activity, ARP-1 cells were pulse treated with 320 μg/ml or 32 μg/ml neoheparin or chondroitin sulfate neoglycan for 5, 15, 30, and 60 minutes at 37° C. and 5% CO₂. The neoglycans were removed and following a 72 hour incubation, cell viability w as determined by MTT assay. Pulse treatment of ARP-1 cells with 320 μg/ml of neoheparin and chondroitin sulfate neoglycan for as short a period as 15 minutes reduces ARP-1 cell viability by 44% and 92%, respectively (FIG. 1G). A 5 minute treatment of ARP-1 cells with chondroitin sulfate neoglycan reduces cell viability by 42%. At the lower dose of 32 μg/ml, a 60 minute pulse with the neoheparin produces a 44% reduction in cell proliferation while the same length pulse with chondroitin sulfate neoglycan produces a 27% reduction in cell viability (FIG. 1H). These results indicate that the neoglycans need not be present continually for a reduction in cancer cell viability. Mechanistically, these results suggest that the neoglycans are affecting cells directly and not simply neutralizing nutrients within the culture media. Moreover, this suggests that even a transient high concentration of neoglycan at the tumor site in vivo could be effective.

### EXAMPLE 10

Neoglycans Induce Apoptosis in Cancer Cells

A reduction in cell viability could reflect slowed growth or could be the result of cell death. Observation of cell cultures treated with either neoglycan reveals morphological changes that strongly suggest the reduction in cell viability is due to cell death (data not shown). To determine directly whether neoglycan treatment of cancer cells promotes apoptosis, ARP-1 cells and MDA-MB-231 cells were treated with media only or media supplemented with GAG chains or neoglycans. Apoptosis was evaluated by two methods, Annexin V-FITC staining, propidium iodide (PI) exclusion and the TUNEL assay.

Annexin-V binds to phosphatidylserine which is on the inner cytoplasmic membrane of healthy cells and on the outer cytoplasmic membrane of apoptotic cells . Treatment of the ARP-1 cells for 48 hours or MDA-MB-231 cells for 96 hours with media only or 32 μg/ml GAG chains resulted in no induction of apoptosis. However, treatment of either cell line with 32 μg/ml neoglycan for the same period of time induces apoptosis as demonstrated by Annexin-V staining on the cell surface (FIG. 2A).

DNA fragmentation is a hallmark of apoptotic cells. ARP-1 and MDA-MB-231 cells were treated with 32 μg/ml of the neoglycans and GAG chains and incubated at 37° C., 5% CO₂ for 48 and 120 hours, respectively. The cells were harvested and DNA fragmentation was detected by the TUNEL assay and the results evaluated by flow cytometry. GAG chain or media only treatment resulted in no DNA fragmentation while treatment of either cell line with either neoglycan resulted in apoptosis as shown by the presence of stained cell populations (FIG. 2B). Collectively the results of two separate assay systems demonstrate that the neoglycans reduce cell viability by inducing apoptosis. Treatment with GAG chains does not induce apoptosis confirming that the neoglycans function differently than native GAG chains.

### EXAMPLE 11

Chondroitin Sulfate Neoglycan Eliminates Established MDA-MB-231 Tumors

NeoCS appears to be more potent than neoheparin in vitro (FIG. 1, Table 1) and neoheparin may exhibit the anticoagulant properties of native heparin. Therefore, chondroitin sulfate neoglycan was used for evaluation of in vivo neoglycan activity. MDA-MB-231 breast cancer tumors were establish in female BALB/c nu/nu mice by subcutaneous injection of 2×10⁶ cells. Two tumors per animal developed and were measured just prior to treatment. A single treatment of 1.6 mg of chondroitin sulfate or chondroitin sulfate neoglycan was administered to separate tumors on the same animal. Following injection of either chondroitin sulfate or neoCS, tumors were electroporated. The size of each tumor was determined 48 hours later and at several subsequent time points.

The results of duplicate experiments demonstrate that a Single dose of chondroitin sulfate neoglycan completely eradicated the MDA-MB-231 breast tumors in every animal by day 5 (FIG. 3). In both experiments, a single chondroitin sulfate neoglycan treated tumor began to regrow by day 13. The chondroitin sulfate neoglycan treated tumors on the other animals did not recur during the one month experimental period.

### EXAMPLE 12

Visualization of Neoglycans and Isolation of an Active Chondroitin Sulfate Neoglycan Fraction

EDAC is used routinely to covalently couple amino groups to carboxyl groups thus combining molecules intra- or intermolecularly depending on the availability of the reactive groups (Sehgal and Vijay 1994). Heparin and chondroitin sulfate contain free carboxyl groups on D-glucuronic acid residues and heparin has limited free amino groups found on N-unsubstituted glucosamine residues (Hook, Riesenfeld et al. 1982). Chondroitin sulfate and heparin each have free amino groups on covalently bound amino acids left from isolation procedures (Lindahl and Roden 1966; Lindahl, Lidholt et al. 1994). This indicates that EDAC modification of these GAG chains should produce a mixture of coupled multimeric GAG molecules.

Equivalent amounts of heparin, neoheparin, CS, and chondroitin sulfate neoglycan were separated by SDS-PAGE and visualized following alcian blue staining (FIG. 4). Comparison of the native GAG chains to the neoglycans shows no obvious difference. Native heparin and chondroitin sulfate are very heterogeneous in size (FIG. 4, Lanes h and CS) and it is therefore conceivable that coupling of the GAG chains is masked. For example, heparin is a large smear ranging in size from less than 14 kDa to greater than 9 7 kDa therefore, even if several low molecular weight chains combined the resulting multimeric complex could be less than 97 kDa and not distinguishable from native heparin. Coupling of different numbers and sizes of GAG chains would not yield a single size product but molecules that vary considerably in size and specific structure. It is therefore, possible that EDAC modification produces coupled GAG chains that are not readily identifiable by SDS-PAGE analysis.

It is possible that contaminating EDAC is contributing to neoglycan killing activity. To address this issue, chondroitin sulfate neoglycan was chondroitin ABC lyase digested in an attempt to block activity and demonstrate that chondroitin sulfate neoglycan specifically kills cancer cells. CS and chondroitin sulfate neoglycan (500 μg each) were digested with 150 μU of lyase and a portion of each along with controls were visualized on SDS-PAGE gels following alcian blue staining (FIG. 5A). The CS-L lane and the neoCS-L lane show high molecular weight smears that remain following lyase digestion. Further lyase treatment failed to digest the remaining chondroitin sulfate or chondroitin sulfate neoglycan (data not shown). Because most of the chondroitin sulfate neoglycan was digested, cell viability experiments were performed to determine if in fact killing activity had been abolished or reduced. ARP-1 cells were treated with digested chondroitin sulfate and chondroitin sulfate neoglycan at a concentration equivalent to 32 μg/ml nondigested material.

Interestingly, the chondroitin sulfate neoglycan activity is not blocked by lyase digestion (FIG. 5B), and in fact, the digested chondroitin sulfate neoglycan is as active as the undigested controls including neoCS-H and neoCS-B. There is no significant difference between the level of killing displayed by chondroitin sulfate neoglycan and lyase digested neoCS. The chondroitin ABC lyase used in these experiments did not affect ARP-1 cell proliferation because cells treated with lyase digested chondroitin sulfate (CS-L) grew a s well as media only treated cells (FIG. 5B). These results suggest either the high molecular weight chondroitin sulfate neoglycan remaining following lyase digestion is active, small digested fragments of the chondroitin sulfate neoglycan are active, or residual EDAC is present and contributing to activity.

To eliminate the possibility that neoglycan preparations contain residual EDAC and to separate high molecular weight chondroitin sulfate neoglycan from low molecular weight digested fragments, chondroitin ABC lyase digested chondroitin sulfate neoglycan was separated by size exclusion chromatography over a Superdex 200 column in buffer containing 6 M guanidine. At this concentration of guanidine everything is denatured and separates based solely on size. Pure fractions of high molecular weight chondroitin sulfate neoglycan were collected, buffer exchanged into H₂O, evaluated by SDS-PAGE and the affect on cell viability w as determined on ARP-1 cells. SDS-PAGE analysis confirmed the separation of different sized high molecular weight components of lyase digested chondroitin sulfate neoglycan ranging in MW from >220 kDa to approximately 97 kDa (FIG. 6A). The 66 kDa band that is evident in fractions 35-40 is bovine serum albumin that is included in the lyase reagent. The concentration of each of these fractions is too low to determine by carbazole reaction although SDS-PAGE analysis of equal volumes of the fractions suggests similar concentrations in the GAG chain containing fractions. ARP-1 cells were treated with equal volumes of fractions 25-42, incubated at 37° C. and 5% CO₂ for 72 hours, and cell viability was determined b y MTT assay. The results indicate that the most active fraction is number 33 and that active molecules are contained within fractions 32-37 (FIG. 6B). EDACis less than 0.2 kDa in size and therefore, any remaining in the chondroitin sulfate neoglycan preparation is removed by column chromatography. These results demonstrate that a contaminant is not producing activity and confirms that the chondroitin ABC lyase resistant high molecular weight chondroitin sulfate neoglycan specifically reduces cell viability.

Discussion

Syndecan-1 is a tumor suppressor that can regulate tumor growth when expressed on the cell surface or when added exogenously to cells. However, tumor growth inhibitory activities are not exclusive to syndecan-1. Other cell surface heparan sulfate proteoglycans such as glypican-1 and betaglycan have similar effects on tumors. Although it has been believed for several years that heparan sulfate proteoglycans play important tumor regulatory roles, no studies have directly addressed their potential for therapies. The idea to use neoglycans as therapeutic agents is novel. This strategy has the potential to produce a relatively simple, inexpensive product that will effectively control tumor growth with little if any negative side effects. These experiments represent a logical next-step for proteoglycan research and, hold promise for the development of new therapies that alone or in combination with other cytostatic agents will arrest or eradicate tumors.

Synthetic neoglycans were constructed in an effort to produce a molecule that would have the tumor suppressor activities of syndecan-1. Syndecan-1 is composed of a protein core bearing several covalently attached heparan sulfate chains, three of which are found in close proximity to each other and form a multimeric complex. These molecular constructions possess the advantage of being relatively easy to construct by simple chemical or other linkage of charged substances. Another advantage is that these can be assembled from abundant, commercially available products such as heparin and chondroitin sulfate. Also, these constructions can be designed to lack immunogenicity in humans. Constructed molecules are an advantage over use of naturally occurring heparan sulfate proteoglycans because the latter are not abundant enough for use therapeutically. Also, recombinant production of proteoglycans is not feasible because they are not properly glycosylated (with heparan sulfate chains). Constructed sulfated arrays also have a decided advantage over the use of heparin alone. For example, heparin alone does not control growth of myeloma tumor cells. Heparin alone also has adverse effects—when injected into humans, it is an anti-coagulant and it stimulates osteoclastogenesis which results in bone loss.

These novel neoglycan constructs possess many uses. For example, the neoglycan constructs may be introduced into humans a s anti-tumor therapy. The constructs may be particularly useful against tumors easily assessable via the blood, e.g., multiple myeloma, which could be treated by intravenous injection. Solid tumors may be treated with direct injection of constructs into the tumor. Following high dose chemotherapy or other purging techniques, sulfated constructs could be used to kill residual tumor cells. These constructs may also bind numerous heparin-binding growth factors or other ligands and regulate their behavior—this could have effects on the pathobiology of numerous diseases (e.g. inflammatory diseases such as rheumatoid arthritis). The constructs may regulate angiogenesis by binding to angiogenic factors such a s bFGF and VEGF, both heparin-binding molecules. It is possible that these constructs could be either pro- or anti-angiogenic in vivo depending on their localization and concentration. Because many viruses and microbes bind to host cells via heparan sulfate, these neoglycans could block their entry into human tissue (e.g. certain HHV, chlymidia). Depending on their concentration in vivo, they could promote or inhibit cell adhesion. It has been shown that heparan sulfate at the cell surface promotes internalization of cationic liposomes into cells. If array constructs when introduced in vivo will bind to cells, this could enhance liposome mediated delivery of genes or other substances into cells. Thus, these neoglycan constructs could be used as a drug delivery tool. Arrays containing an attached drug may bind to heparan binding receptors on the cell surface thereby delivering them to the cell. cDNA constructs coding for small peptides that will be substituted with heparan sulfate chains could be prepared. Such genes could be transferred to cells in vivo, resulting in cells that secrete therapeutic amounts of multi-valent heparan sulfate molecules.

It is possible that the neoglycans of the present invention will have anti-coagulant effects in vivo. If so, they can be fractionated and fractions analyzed for their anti-coagulant effect. The anti-coagulant effects of heparin and heparan sulfate are mediated by a very specific carbohydrate sequence, therefore it is likely that other biological effects of heparin will not be lost by removing the anti-coagulant fraction. The effects of heparin constructs on normal cells are unknown, but could possibly be detrimental.

GAGGAGGAG EDAC coupling reactions are widely used to conjugate carboxyl and amino group containing molecules (Bauminger and Wilchek 1980). Heparin is composed of long variable length, unbranched chains of a repeating disaccharide unit of D-glucosamine and D-glucuronic acid or L-iduronic acid. CS is also a long, variable length, unbranched chain of repeating disaccharide units consisting of a D-galactosamine and a D-glucuronic acid residue. Both GAG chains are epimerized and sulfate substituted at varying points along the chains ((Bernfield, Gotte et al. 1999). The number 6 carbon of the glucuronic acid components of both GAG chains contains a free carboxyl group. Heparin that has been isolated from pig intestinal mucosa contains a limited number of N-nonsubstituted glucosamine residues and therefore a limited number of free amino groups (Hook, Riesenfeld et al. 1982). Heparin and chondroitin sulfate used for the production of neoglycans contain free amino groups on covalently bound amino acids remaining from conventional isolation procedures (Hook, Riesenfeld et al. 1982). Therefore, EDAC modification of heparin and chondroitin sulfate likely results in molecules composed of coupled GAG chains thereby producing multimeric GAG complexes similar to the multimeric array of heparan sulfate found on syndecan-1.

Molecules produced from coupling different size and number of GAG chains would be extremely divergent in size and therefore, appear as a smear on an SDS-PAGE gel. EDAC modified heparin and chondroitin sulfate have a smeared electrophoretic pattern that is similar to the native GAG chains and does not reveal a clear shift in size and therefore, an obvious coupling of the GAG chains (FIG. 4). Due to the heterogeneity in size, native heparin and chondroitin sulfate chains could mask coupled neoglycan molecules. Small GAG chains could be coupled yet not exceed the size of large GAG chains and therefore, the neoglycans would not be distinguishable from native GAG chains by SDS-PAGE analysis. Although presently, the structure of the neoglycans is not clear, isolation of the active chondroitin sulfate neoglycan fractions facilitates future studies for structure determination.

EDAC modification of GAG chains produces molecules with strikingly different activities as compared to the native GAG chains.

Treatment of cell lines with native heparin and chondroitin sulfate does not reduce cell growth and in fact it enhances cell growth in some cases (FIG. 1). Heparin and chondroitin sulfate do not induce apoptosis in ARP-1 and MDA-MB-231 cancer cell lines (FIG. 2). In great contrast to heparin and CS, the neoheparin and chondroitin sulfate neoglycan severely reduce cell viability in normal and cancer cell lines and induce apoptosis in the ARP-1 multiple myeloma cell line and the MDA-MB-231 breast cancer cell line. Interestingly, the activity of the neoglycans can not be blocked by the addition of excessive amounts of GAG chains. Treating ARP-1 -cells with 10 fold higher concentrations of native GAG chains does not change the killing affect of the neoglycans (data not shown) a result which highlights the different functions of GAG chains and neoglycans. The contrasting functions of the native chondroitin sulfate and the chondroitin sulfate neoglycan are also evident in the results of in vivo experiments. NeoCS eradicates breast cancer tumor burden in established tumors in nude mice whereas, chondroitin sulfate does not reduce the tumor burden (FIG. 4).

Ideally, a therapeutic agent should selectively act on the appropriate target in a expeditious manner. Neoglycan (32 μg/ml) pulse treatment for one hour results in a reduction in ARP-1 cell viability while at a higher concentration (320 μg/ml) a pulse as short as 5-15 minutes results in a 44-92% reduction in cell viability (FIGS. 1G and 1H). This finding suggests that the mechanism of activity of the neoglycans involves direct interaction with the cells a s opposed to an indirect means of killing such as depletion of essential components in the media. The ability to produce an effect quickly and irreversibly is fortuitous because many anticancer therapeutics are cleared rapidly or metabolized.

The neoglycans are potent inhibitors of multiple myeloma and breast cancer cell line proliferation however, the neoglycans also inhibit the growth of several normal cell lines and activated primary PBMCs (FIGS. 2A-2G). Notably, chondroitin sulfate neoglycan treatment of established breast cancer tumors in nude mice showed alleviated tumor burden with no obvious toxicity to surrounding tissue or the whole animal. Therefore, the less than desirable selectivity observed in vitro may be an artifact of rapidly dividing laboratory cultures. The isolation of volumes of the active fraction of chondroitin sulfate neoglycan along with structure determination will provide the necessary information for the design and production of more selective neoglycan analogues while maintaining the already expeditious activity.

The production of neoglycans is a simple protocol involving only two reagents, GAG chains and EDAC. EDAC is less than 0.2 kDa in size and is removed by spin column filtration. However, it is possible that residual EDAC is left in the neoglycan preparations and is contributing to activity. Chondroitin sulfate neoglycan was digested with chondroitin ABC lyase in an attempt to block activity and thereby demonstrate that the chondroitin sulfate neoglycan not residual EDAC produces the killing activity. Surprisingly, digestion of chondroitin sulfate neoglycan reveals a minor portion of chondroitin sulfate neoglycan that is recalcitrant to digestion and retains the ability to reduce cell viability (FIG. 5). These results suggest that lyase digestion of chondroitin sulfate neoglycan may be a useful method for the isolation of the active chondroitin sulfate neoglycan fraction however, these results did not eliminate the possibility that contaminating EDAC is present. Therefore, lyase digested chondroitin sulfate neoglycan was denatured in 6M guanidine and separated from any residual EDAC by column chromatography.

Treatment of ARP-1 cells with the column fractions indicates a reduction of cell viability by specific chondroitin sulfate neoglycan containing fractions and no affect by other fractions (FIG. 6B). These results confirm that the chondroitin sulfate neoglycan specifically is responsible for the observed reduction in multiple myeloma cell viability. These results also demonstrate the presence of active and inactive chondroitin sulfate neoglycan fractions (FIG. 6B). Although chondroitin sulfate neoglycan containing fractions were active, the GAG concentration was too low to determine. Therefore, the removal of inactive portions of chondroitin sulfate neoglycan vastly lowers the concentration required to kill tumor cells.

In conclusion, the instant invention describes the production and the anticancer properties of novel therapeutic agents called neoglycans. The mechanism of neoglycan antitumor activity is wholly unknown although, it is likely that a direct affect occurs because a short exposure to neoheparin or chondroitin sulfate neoglycan irreversibly kills cells. The isolation of active chondroitin sulfate neoglycan fractions provides the foundation for future studies of the structure and function of neoglycans and for the optimization of neoglycan activities. The discovery of neoglycan anticancer affects is especially exciting because by exploiting the structural and functional diversity of the many members of the GAG family a whole new class of anticancer agents can be developed.

The following references were cited herein:

Alexanian, R., B. Barlogie, et al. (1986). “High-dose glucocorticoid treatment of resistant myeloma.” Ann Intern Med 105(1): 8-11.

Arribas, J., L. Coodly, et al. “Diverse cell surface protein ectodomains are shed by a system sensitive to metalloprotease inhibitors.”

Bauminger, S. and M. Wilchek (1980). The use of carbodiimides in the preparation of immunizing conjugates. Methods of Enzymology. H. V. Vunakis and J. H. Langone. New York, Academic Press, Inc. 70: 151-159.

Bernfield, M., M. Gotte, et al. (1999). “Functions of cell surface heparan sulfate proteoglycans.” Annu Rev Biochem 68: 729-77.

Bijsterveld, N. R., R. Hettiarachchi, et al. (1999). “Low-molecular weight heparins in venous and arterial thrombotic disease.” Thromb Haemost 82 Suppl 1: 139-47.

Blumenkrantz, N. and G. Asboe-Hansen (1973). “New method for quantitative determination of uronic acids.” Anal Biochem 54(2): 484-9.

Burk, K. H., B. Drewinko, et al. (1978). “Establishment of a human plasma cell line in vitro.” Cancer Res 38(8): 2508-13.

Carey, D. J., R. C. Stahl, et al. (1994). “Syndecan-1 expressed in Schwann cells causes morphological transformation and cytoskeletal reorganization and associates with actin during cell spreading.” J Cell Biol 124(1-2): 161-170.

Collen, A., S. M. Smorenburg, et al. (2000). “Unfractionated and low molecular weight heparin affect fibrin structure and angiogenesis in vitro.” Cancer Res 60(21): 6196-200.

Dhodapkar, M. V., E. Abe, et al. (1998). “Syndecan-1 is a multifunctional regulator of myeloma pathobiology: control of tumor cell survival, growth, and bone cell differentiation.” Blood 91(8): 2679-88.

Filisetti-Cozzi, T. M. and N. C. Carpita (1991). “Measurement of uronic acids without interference from neutral sugars.” Anal Biochem 197(1): 157-62.

Folkman, J. and Y. Shing (1992). “Control of angiogenesis by heparin and other sulfated polysaccharides.” Adv Exp Med Biol 313: 355-64.

Hejna, M., M. Raderer, et al. (1999). “Inhibition of metastases b y anticoagulants.” J Natl Cancer Inst 91(1): 22-36.

Hook, M., J. Riesenfeld, et al. (1982). “N-[3H]Acetyl-labeling, a convenient method for radiolabeling of glycosaminoglycans.” Anal Biochem 119(2): 236-45.

Hooper, N. M., E. H. Karran, et al. (1997). “Membrane protein secretases.” Biochem J 321(Pt 2): 265-79.

Inki, P. and M. Jalkanen (1996). “The role of syndecan-1 in malignancies.” Ann Med 28(1): 63-7.

Inki, P., H. Joensuu, et al. (1994). “Association between syndecan-1 expression and clinical outcome in squamous cell carcinoma of the head and neck.” Br J Cancer 70(2): 319-323.

Kato, M., S. Saunders, et al. (1995). “Loss of cell surface syndecan-1 causes epithelia to transform into anchorage-independent mesenchyme-like cells.” Molecular Biol Cell 6(5): 559-576.

Kumar-Singh, S., W. Jacobs, et al. (1998). “Syndecan-1 expression in malignant mesothelioma: correlation with cell differentiation, WT1 expression, and clinical outcome.” J Pathol 186(3): 300-305.

Leppa, S., P. Harkonen, et al. (1991). “Steroid-induced epithelial-fibroblastic conversion associated with syndecan suppression in S115 mouse mammary tumor cells.” Cell Regulation 2(1): 1-11.

Leppa, S., M. Mali, et al. (1992). “Syndecan expression regulates cell morphology and growth of mouse mammary epithelial tumor cells.” PNAS 89(3): 932-936.

Leppa, S., K Vleminckx, et al. (1996). “Syndecan-1 Expression In Mammary Epithelial Tumor Cells Is E-Cadherin-Dependent.” J Cell Science 109(Part 6): 1393-1403.

Lesoon-Wood, L. A., W. H. Kim, et al. (1995). “Systemic gene therapy with p53 reduces growth and metastases of a malignant human breast cancer in nude mice.” Hum Gene Ther 6(4): 395-405.

Liebersbach, B. F. and R. D. Sanderson (1994). “Expression of syndecan-1 inhibits cell invasion into type I collagen.” J Biol Chem 269(31): 20013-20019.

Lind, T., F. Tufaro, et al. (1998). “The putative tumor suppressors EXT1 and EXT2 are glycosyltransferases required for the biosynthesis of heparan sulfate.” J Biol Chem 273(41): 26265-8.

Lindahl, U., K. Lidholt, et al. (1994). “More to “heparin” than anticoagulation.” Thromb Res 75(1): 1-32.

Lindahl, U. and L. Roden (1966). “The chondroitin 4-sulfate-protein linkage.” J Biol Chem 241(9): 2113-9.

Mali, M., H. Andtfolk, et al. (1994). “Suppression of tumor cell growth by syndecan-1 ectodomain.” J Biol Chem 269(45): 27795-27798.

Mali, M., H. Andtfolk, et al. (1994). “Suppression of tumor cell growth by syndecan-1 ectodomain.” J Biol Chem 269(45): 27795-8.

Matsumoto, A., M. Ono, et al. (1997). “Reduced expression of syndecan-1 in human hepatocellular carcinoma with high metastatic potential.” Int J Cancer 74(5): 482-491.

Mbalaviele, G., C. R. Dunstan, et al. (1996). “E-cadherin expression in human breast cancer cells suppresses the development of osteolytic bone metastases in an experimental metastasis model.” Cancer Res 56(17): 4063-70.

McCormick, C., Y. Leduc, et al. (1998). “The putative tumour suppressor EXT1 alters the expression of cell-surface heparan sulfate.” Nat Genet 19(2): 158-61.

Nackaerts, K., E. Verbeken, et al. (1997). “Heparan sulfate proteoglycan expression in human lung-cancer cells.” Int J Cancer 74(3): 335-345.

Oh, E. S., A. Woods, et al. (1997). “Syndecan-4 proteoglycan regulates the distribution and activity of protein kinase C.” J Biol Chem 272(13): 8133-6.

Park, P. W., O. Reizes, et al. (2000). “Cell surface heparan sulfate proteoglycans: selective regulators of ligand-receptor encounters.” J Biol Chem 275(39): 29923-6.

Pieters, R., D. R. Huismans, et al. (1988). “Adaptation of the rapid automated tetrazolium dye based (MTT) assay for chemosensitivity testing in childhood leukemia.” Cancer Lett 41(3): 323-32.

Pulkkinen, J. O., M. Penttinen, et al. (1997). “Syndecan-1—a New Prognostic Marker In Laryngeal Cancer.” Acta Oto-Laryngologica 117(2): 312-315.

Rapraeger, A. C. (1993). “The coordinated regulation of heparan sulfate, syndecans and cell behavior.” Curr. Opin. Cell Biol. 5: 844-853.

Reiland, J., V. L. Ott, et al. (1996). “Pervanadate activation of intracellular kinases leads to tyrosine phosphorylation and shedding of syndecan-1.” Biochem J 319(Part 1): 39-47.

Ridley, R. C., H, Xiao, et al. (1993). “Expression of syndecan regulates human myeloma plasma cell adhesion to type I collagen.” Blood 81(3): 767-74.

Sehgal, D. and I. K. Vijay (1994). “A method for the high efficiency of water-soluble carbodiimide-mediated amidation.” Anal Biochem 218(1): 87-91.

Siragusa, S., B. Cosmi, et al. (1996). “Low-molecular-weight heparins and unfractionated heparin in the treatment of patients with acute venous thromboembolism: results of a meta-analysis.” Am J Med 100(3): 269-77.

Smorenburg, S. M. and C. J. Van Noorden (2001). “The complex effects of heparins on cancer progression and metastasis in experimental studies.” Pharmacol Rev 53(1): 93-105.

Tanaka, Y., K. Kimata, et al. (1998). “Modulation of cytokine function by heparan sulfate proteoglycans: sophisticated models for the regulation of cellular responses to cytokines.” Proc Assoc Am Physicians 110(2): 118-25.

Turnbull, J., A. Powell, et al. (2001). “Heparan sulfate: decoding a dynamic multifunctional cell regulator.” Trends Cell Biol 11(2): 75-82.

Vincent, T., M. Jourdan, et al. (2001). “Hyaluronic Acid Induces Survival and Proliferation of Human Myeloma Cells through an Interleukin-6-mediated Pathway Involving the Phosphorylation of Retinoblastoma Protein.” J Biol Chem 276(18): 14728-36.

Wiksten, J. P., J. Lundin, et al. (2001). “Epithelial and stromal syndecan-1 expression as predictor of outcome in patients with gastric cancer.” Int J Cancer 95(1): 1-6.

Zeng, C., B. P. Toole, et al. (1998). “Inhibition of tumor growth in vivo by hyaluronan oligomers.” Int J Cancer 77(3): 396-401.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

TABLE 1
Fifty percent inhibitory concentration of neoglycans for the reduction of cancer and normal cell viability in vitro.

| | | | | | | nH CI^{b} | nCS | nCS CI^{b} |
|---|---|---|---|---|---|---|---|---|
| | Tumor- | | | | nH IC50^{a} | range | IC50 | range |
| Organism | genicity | Tissue | Morphology | Designation | (ug/ml) | (ug/ml) | (ug/ml) | (ug/ml) |
| human | + | multiple myeloma | lymphoblast, | ARK | 27.42 | 21.96 | 24.92 | 18.03 |
| | | | plasma cell | | | 34.24 | | 34.45 |
| human | + | multiple myeloma | lymphoblast, | ARP-1 | 21.94 | 18.18 | 14.79 | 12.13 |
| | | | plasma cell | | | 26.48 | | 18.06 |
| human | + | multiple myeloma | lymphoblast, | CAG | 16.02 | 14.09 | 13.65 | 11.88 |
| | | | plasma cell | | | 18.21 | | 15.68 |
| human | + | multiple myeloma | lymphoblast, | U266 | 32.00 | 26.44 | 19.05 | 16.83 |
| | | | plasma cell | | | 38.73 | | 21.55 |
| human | + | plasma cell | lymphoblast, | ARH-77 | 19.74 | 12.18 | 21.87 | 9.06 |
| | | leukemia | plasma cell | | | 31.99 | | 52.80 |
| human | ± | breast | epithelial | HBL-100 | 8.28 | 5.89 | 6.81 | 2.92 |
| | | adenocarcinoma | | | | 11.65 | | 15.90 |
| human | + | breast | epithelial | Hs578t | 2.34 | 1.67 | 3.65 | 3.20 |
| | | adenocarcinoma | | | | 3.28 | | 4.16 |
| human | + | breast | epithelial | MCF-7 | 11.49 | 3.52 | 6.55 | 4.26 |
| | | adenocarcinoma | | | | 37.49 | | 10.06 |
| human | + | breast | epithelial | MDA-MB- | 17.70 | 14.59 | 5.00 | 3.28 |
| | | adenocarcinoma | | 231 | | 21.28 | | 7.61 |
| human | + | breast | epithelial | MDA-MB- | 17.10 | 12.25 | 6.82 | 4.51 |
| | | adenocarcinoma | | 435 | | 23.86 | | 10.29 |
| human | + | breast | epithelial | MDA-MB- | 4.21 | 2.66 | 3.86 | 2.80 |
| | | adenocarcinoma | | 436 | | 6.68 | | 5.33 |
| human | − | breast | epithelial | MCF-10A | 4.16 | 2.76 | 4.29 | 2.67 |
| | | | | | | 6.29 | | 6.89 |
| human | − | breast | epithelial | MCF-10F | | | | |
| chinese | − | ovary | epithelial | CHO-K1 | 3.38 | 2.23 | 3.33 | 3.13 |
| hamster | | | | | | 5.13 | | 3.53 |
| human | − | breast | epithelial | Hs578BST | | | | |
| canine | − | kidney | epithelial | MDCK | 15.98 | 4.97 | 10.64 | 7.63 |
| | | | | | | 51.36 | | 14.83 |
| murine | − | embryo | epithelial | NIH3T3 | 7.73 | 3.42 | 15.74 | 12.22 |
| | | | | | | 17.50 | | 20.29 |
| murine | − | breast | epithelial | NMuNG | 6.29 | 4.26 | 5.87 | 4.60 |
| | | | | | | 9.29 | | 7.50 |
| human | − | blood | lymphocyte, | PBMC | 3.94 | 3.22 | 4.21 | 3.64 |
| | | | monocyte | | | 4.82 | | 4.86 |

^{a}Estimated IC50 values were determined following cell viability assays by use of a compound nonlinear degradation model based on the 3 parameter logistic. Calculations are based on triplicat wells from duplicate experiments.
^{b}95% confidence interval.

What is claimed is:

1. A neoglycan capable of inhibiting tumor cell growth, wherein said neoglycan comprises glycosylaminoglycan (GAG) chains chemically modified with a carbodiimide.

2. The neoglycan of claim 1, wherein said carbodiimide is 1-ethyl-3-(3-dimethylaminopropyl)carbodiimide.

3. The neoglycan of claim 1, wherein said GAG chains are selected from the group consisting of heparin, heparan sulfate, chondroitin sulfate, hyaluronic acid, dextran, dextran sulfate and synthetically produced GAG-like molecules.

4. The neoglycan of claim 3, wherein said neoglycan is neoheparin comprising EDAC modified heparin chains.

5. The neoglycan of claim 3, wherein said neoglycan is neo-chondroitin sulfate comprising EDAC modified chondroitin sulfate chains.

6. A method of producing the neoglycan of claim 1, comprising the steps of:
treating a solution of a GAG with carbodiimide;
subjecting said solution to centrifugation to remove any precipitate; and,
buffer exchanging said solution into water.

7. The method of claim 6, wherein said carbodiimide is 1-ethyl-3-(3-dimethylaminopropyl)carbodiimide.

8. The method of claim 6, wherein said GAG chains are selected from the group consisting of heparin, heparan sulfate, chondroitin sulfate, hyaluronic acid, dextran and dextran sulfate or other synthetically produced GAG-like molecules.

9. A pharmaceutical composition comprising the neoglycan of claim 1 and a pharmaceutically acceptable carrier.

10. A method of inhibiting tumor growth, comprising the step of:
exposing said tumor to the pharmaceutical compound of claim 9.

11. The method of claim 10, wherein said method induces apoptosis of the cells of said tumor.

12. The method of claim 10, wherein said tumor is selected from the group consisting of breast cancer, multiple myeloma and others.
